(19) United States
(12) Patent Application Publication
(10) Pub. No.: US 2005/0048018 A1
(43) Pub. Date: Mar. 3, 2005

(54) Methods for preventing reversion of relaxed keratinous fibers and for relaxing keratinous fibers

(76) Inventors: Natasha Fadeeva, Clark, NJ (US); Nghi Van Nguyen, Edison, NJ (US); David W. Cannell, Plainfield, NJ (US)

Correspondence Address: Finnegan, Henderson, Farabow,;Garrett & Dunner, L.L.P., 1300 I Street, N.W., Washington, DC, 20005-3315, US

(21) Appl. No.: 10/650,822

(22) Filed: Aug. 29, 2003

(51) Int. Cl. A61K 7/06; A61K 7/11

(52) U.S. Cl. 424/70.13

(57) ABSTRACT

Methods for preventing reversion of at least one relaxed keratinous fiber and for relaxing at least one keratinous fiber comprising applying compositions comprising at least one sugar chosen from C₃ to C₅ monosaccharides and heating at least one keratinous fiber.

The present invention relates to methods for preventing reversion of at least one relaxed keratinous fiber and to methods and kits for relaxing at least one keratinous fiber, including human keratinous fibers, comprising at least one sugar chosen from C₃ to C₅ monosaccharides and derivatives thereof.

Hair fiber is a keratinous material which comprises proteins (polypeptides). Many of the polypeptides in hair fibers are bonded together or cross-linked with disulfide bonds (—S—S—). A disulfide bond may be formed from the reaction of two sulfhydryl groups (—SH), one on each of two cysteine residues, which results in the formation of a cystine residue. A cystine residue comprises a cross-link of the formula —CH₂—S—S—CH₂— between 2 polypeptides. While there are other types of bonds which occur between the polypeptides in hair fibers, such as ionic (salt) bonds, the permanent curling or shape of the hair is essentially dependent on the disulfide bonds of cystine residues.

In today's market, there is an increasing demand for the hair care products referred to as “hair relaxers,” which can relax or straighten naturally curly or kinky hair. Straightening or relaxing the curls of very curly hair may increase the manageability and ease of styling of such hair. Hair relaxers may either be applied in a hair salon by a professional or in the home by the individual consumer.

Generally, hair relaxing processes are chemical processes which may alter the aforementioned disulfide bonds between polypeptides in hair fibers and may form lanthionine residues [S[CH₂CH(NH—)(CO—)]₂]. Thus, the term “lanthionizing” is used when one skilled in the art refers to the relaxing of keratinous fibers. For example, hair fibers may be relaxed or straightened by disrupting the disulfide bonds of the hair fibers with an alkaline or reducing agent. The chemical disruption of disulfide bonds with such an agent is generally combined with mechanical straightening of the hair, such as combing, and straightening generally occurs due to changes in the relative positions of neighboring polypeptide chains within the hair fiber. This reaction is generally terminated by rinsing and/or application of a neutralizing composition.

Most frequently, commercial relaxing compositions are in the form of gels or emulsions that contain varying proportions of strong water-soluble bases, such as sodium hydroxide (NaOH), or of compositions that contain slightly-soluble metal hydroxides, such as calcium hydroxide (Ca(OH)₂), which can be converted in situ to soluble bases, such as guanidine hydroxide. Traditionally, the two main hair relaxers used in the hair care industry for generating hydroxide ions are referred to as “lye” (lye=sodium hydroxide) relaxers and “no lye” relaxers, such as potassium hydroxide, lithium hydroxide, and calcium hydroxide.

Reducing agents such as compounds comprising at least one thiol group may also relax or straighten hair by disrupting disulfide bonds of the hair fibers. More commonly, reducing agents, such as thioglycolates, sulfites, cysteines and their derivatives, are used for texturizing purposes in hair straightening or relaxing compositions.

The reducing step is generally a reversible reduction reaction of disulfide bonds within a keratinous fiber. For example, the reaction of a thiol-containing reducing agent, RSH, with hair can be represented by the following reaction scheme, wherein k represents the keratin protein chain of a keratinous fiber:
k-CH₂—S—S—CH₂-k+RSH [symbol] k-CH₂—S—S—R+k-CH₂—SH (I)
k-CH₂—S—S—R+RSH [symbol] k CH₂—SH+RS—SR (II)

Generally, the disulfide product, RS—SR, and any residual reducing agent, RSH, are rinsed from the hair, and then the disulfide bonds are restored in the neutralizing step. The neutralizing step can be represented by the following reaction scheme:

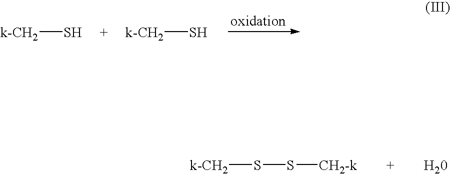

With both hydroxide-containing alkaline agents and thiol-containing reducing agent, a high concentration of reducing agent may result in hair damage and a low concentration may result in reversion of the hair to its original curly state. Accordingly, a composition which prevents the reversion of relaxed hair is desirable.

Sugars and sugar derivatives are one class of the countless number of compounds that have been added to hair care compositions. Documented uses of sugars in hair care compositions include: the use of glucose to improve the tactile and elastic properties of natural hair (Holienberg and Mueller, SOFW J. 121(2) (1995)); the use of glucose for hair damage prophylaxis and damaged hair repair (Hollenberg & Matzik, Seifen, Oele, Fette, Wachase 117(1) (1991)); the use of glucose in shampoos (J04266812, assigned to Lion Corp.); the use of trehalose for moisture retention (J06122614, assigned to Shiseido Co. Ltd.); a composition for the lanthionization of hair comprising a sugar (U.S. Pat. Nos. 5,348,737 and 5,641,477, assigned to Avlon Ind. Inc.); the incorporation of xylobiose into cosmetic compositions to provide enhanced moisture retention and reduce excessive roughness and dryness of the skin and hair (U.S. Pat. No. 5,660,838, assigned to Suntory Ltd.); a composition for the regeneration of hair split-ends that contains at least one mono- or di-saccharide (U.S. Pat. No. 4,900,545, assigned to Henkel); hair care compositions to improve hair strength, hold and volume that contain C₅ to C₆ carbohydrates such as glucose; the use of fucose in a hair treatment to prevent split ends (DE29709853, assigned to Goldwell GMBH); and the use of saccharides in a shampoo to improve combing properties and control hair damage (J09059134, assigned to Mikuchi Sangyo KK).

In essence, sugars have been applied to hair for many reasons from moisturizing to enhancing hair growth (J10279439, assigned to Kureha Chem. Ind. Co. Ltd.). Clearly, however, not all sugars are the same and not all sugars impart the same properties when applied to a keratinous fiber.

The inventors have envisaged the application to at least one relaxed keratinous fiber of at least one composition comprising at least one sugar chosen from C₃ to C₅ monosaccharides and derivatives thereof. In particular, the inventors have discovered that methods comprising applying these compositions to at least one relaxed keratinous fiber and heating the at least one relaxed keratinous fiber prevent reversion of the relaxed hair to a curly or wavy state.

Thus, to achieve at least one of these and other advantages, the present invention, in one aspect, provides a method for relaxing keratinous fibers without substantial reversion to the original curly state of the hair using relaxing compositions and compositions comprising at least one sugar chosen from C₃ to C₅ monosaccharides and derivatives thereof.

Thus, the present invention provides, in one embodiment, a method for preventing reversion of at least one relaxed keratinous fiber comprising (i) applying to at least one relaxed keratinous fiber a composition comprising at least one sugar chosen from C₃ to C₅ monosaccharides and derivatives thereof; and (ii) heating the at least one relaxed keratinous fiber, wherein the at least one sugar is present in an amount effective to prevent reversion of the at least one relaxed keratinous fiber, and further wherein the composition is applied prior to or during heating. In an embodiment, the inventive method further comprises wetting the at least one relaxed keratinous fiber with water prior to application of the composition. In an embodiment, the inventive method further comprises rinsing the at least one relaxed keratinous fiber subsequent to heating. In an embodiment, the composition is heat-activated.

The present invention also provides a method for lanthionizing keratinous fibers to achieve relaxation of at least one keratinous fiber comprising (i) applying at least one relaxing composition to at least one keratinous fiber for a sufficient period of time to lanthionize said at least one keratinous fiber; (ii) rinsing the at least one relaxed keratinous fiber; (iii) applying to the at least one relaxed keratinous fiber a composition comprising at least one sugar chosen from C₃ to C₅ monosaccharides and derivatives thereof; and (iv) heating the at least one relaxed keratinous fiber, wherein the at least one sugar is present in an amount effective to prevent reversion of the at least one relaxed keratinous fiber, and further wherein the composition is applied prior to or during heating. In an embodiment, the at least one relaxing composition comprises at least one reducing agent chosen from hydroxide compounds, thiols, sulfites, and derivatives thereof.

Further, the present invention also provides for a multicomponent kit for lanthionizing keratinous fibers, wherein the kit comprises at least two compartments. A first compartment of the kit contains at least one relaxing composition and a second compartment contains at least one sugar chosen from C₃ to C₅ monosaccharides and derivatives thereof.

Certain terms used herein are defined below:

As used herein, “at least one” means one or more and thus includes individual components as well as mixtures/combinations.

“Heating” refers to the use of elevated temperature (i.e., above 45° C.). In one embodiment, “heating refers to the use of temperatures above 100° C., such as above 200° C. In one embodiment, the heating in the inventive method may be provided by directly contacting the keratinous fibers with a heat source, e.g., by heat styling of the keratinous fibers. Non-limiting examples of heat styling by direct contact with the keratinous fibers include flat ironing, and curling methods using elevated temperatures (such as, for example, curling with a curling iron and/or hot rollers). In another embodiment, the heating in the inventive method may be provided by heating the keratinous fibers with a heat source which may not directly contact the keratinous fibers. Non-limiting examples of heat sources which may not directly contact the keratinous fibers include blow dryers, hood dryers, heating caps and steamers.

“Keratinous fibers” as defined herein may be human keratin fibers, and may be chosen from, for example, hair.

“Lanthionizing,” as used herein, refers to the formation of at least one lanthionine residue, which may accomplish any level of relaxation, for example, from slight relaxing to straightening.

“Relaxation,” “relaxing,” and “relaxed” as used herein, includes any level of relaxing resulting from lanthionization of hair, for example, from slight relaxing to straightening.

A “relaxing composition,” as used herein, refers to compositions, also called “hair relaxers,” which can relax or straighten naturally curly, wavy and/or kinky hair. Relaxing compositions may either be applied in a hair salon by a professional or in the home by the individual consumer.

“Reversion” of at least one relaxed keratinous fiber, as used herein, refers to at least partial return of the at least one relaxed keratinous fiber hair toward the original curly, wavy, and/or kinky state which it was in prior to relaxation.

It is to be understood that both the foregoing general description and the following detailed description are exemplary and explanatory only and are not restrictive of the invention, as claimed. Reference will now be made in detail to exemplary embodiments of the present invention.

As described above, sugars have been used in hair care compositions and other treatments for their moisture retaining properties. However, it was unexpectedly discovered by the present inventors that, in addition to retaining moisture, a certain class of sugars had other properties that made them particularly desirable for use on relaxed keratinous fibers. In particular with respect to hair, compositions comprising at least one sugar chosen from C₃ to C₅ monosaccharides were found to prevent reversion of relaxed hair. Thus, these compositions may impart to at least one keratinous fiber a higher degree of relaxation or a more permanent degree or relaxation even after exposure to high humidity, which is known to cause reversion of relaxed hair. This is particularly true when the compositions are applied to the hair, and the hair is then heated.

Thus, the invention provides a method for preventing reversion of at least one relaxed keratinous fiber comprising (i) applying to at least one relaxed keratinous fiber a composition comprising at least one sugar chosen from C₃ to C₅ monosaccharides and derivatives thereof; and (ii) heating the at least one relaxed keratinous fiber, wherein the at least one sugar is present in an amount effective to prevent reversion of the at least one relaxed keratinous fiber, and further wherein the composition is applied prior to or during heating. In an embodiment, the inventive method further comprises wetting the at least one keratinous fiber with water prior to application. In an embodiment, the inventive method further comprises rinsing the at least one keratinous fiber subsequent to heating. In an embodiment, the composition is heat-activated. The composition may further comprise at least one additional sugar.

In an embodiment, the at least one relaxed keratinous fiber is heated at 130° C. for 10 seconds. In another embodiment, the at least one relaxed keratinous fiber is heated at 180° C. for 3 seconds.

The C₃ to C₅ monosaccharides according to the present invention may be chosen from any triose, tetrose and pentose. (Nomenclature: C3—triose, C4—tetrose, C5—pentose). Further, the C₃ to C₅ monosaccharides can be chosen from the D-form, L-form and mixtures of any of the foregoing. Non-limiting examples of C₃ to C₅ monosaccharides include aldopentoses (such as xylose, arabinose, lyxose, and ribose), ketopentoses (such as ribulose and xylulose), aldotetroses (such as erythrose and treose), ketotetroses (such as erythrulose), aldotrioses (such as glyceraldehyde) and ketotrioses (such as dihydroxyacetone). The C₃ to C₅ monosaccharides may be chosen from C₃ to C₅ monosaccharides comprising aldehyde groups (aldoses), furanoses and other ring structures. The C₃ to C₅ monosaccharides may be substituted or unsubstituted.

Derivatives of C₃ to C₅ monosaccharides may be used as the at least one sugar of the present invention. For example, ammonias or primary amines may react with the aldehyde or ketone group of a sugar to form an imine derivative (i.e., a compound containing the functional group C═N). These imine compounds are sometimes also referred to as Schiff bases. Other non-limiting examples of derivatives of C₃ to C₅ monosaccharides are hemiacetal derivatives of C₃ to C₅ monosaccharides, hemiketal derivatives of C₃ to C₅ monosaccharides and any oxidized derivatives of C₃ to C₅ monosaccharides. These derivatives may be formed, for example, from the reaction of the aldehyde or ketone group of a sugar with an alcohol. Other exemplary derivatives of C₃ to C₅ monosaccharides may also include, but are not limited to, oligosaccharides derived from C₃ to C₅ monosaccharides. As previously mentioned, the at least one sugar chosen from C₃ to C₅ monosaccharides may be substituted or unsubstituted. Thus, in one embodiment, the derivatives of C₃ to C₅ monosaccharides may be substituted or unsubstituted.

According to the present invention, the at least one sugar is present in the composition in an amount generally ranging from 0.01% to 10% by weight relative to the total weight of the composition, such as from 0.1% to 5% by weight. According to the present invention, an “amount effective to prevent reversion” of the at least one sugar may be determined according to Examples 1-6.

The compositions of the present invention as well as those of the inventive methods may further comprise at least one additional sugar which is different from the at least one sugar chosen from C₃ to C₅ monosaccharides. The at least one additional sugar may, for example, aid in moisture retention.

The at least one additional sugar may be chosen from any sugar, carbohydrate or carbohydrate moiety. Non-limiting examples of the at least one additional sugar are monosaccharides, which include, but are not limited to, three to seven carbon sugars such as pentoses (for example, ribose, arabinose, xylose, lyxose, ribulose, and xylulose) and hexoses (for example, allose, altrose, glucose, mannose, gulose, idose, galactose, talose, sorbose, psicose, fructose, and tagatose); oligosaccharides such as disaccharides (such as maltose, sucrose, cellobiose, trehalose and lactose); and polysaccharides such as starch, dextrins, cellulose and glycogen. In another embodiment, the at least one additional sugar is chosen from any aldoses and ketoses. Further, the at least one additional sugar may be substituted or unsubstituted.

According to the present invention, the at least one additional sugar is present in the composition in an amount generally ranging from 0.01% to 10% by weight relative to the total weight of the composition, such as from 0.1% to 5% by weight.

The compositions of the present invention as well as those used in the methods of the present invention may be in the form of a liquid, an oil, a paste, a stick, a dispersion, an emulsion, a lotion, a gel, or a cream. These inventive compositions may further comprise at least one solvent. Non-limiting examples of the at least one solvent include water and organic solvents. Non-limiting examples of organic solvents include C₁-C₄ alkanols, such as ethanol and isopropanol; glycerol; glycols and glycol ethers, such as 2-butoxyethanol, propylene glycol, propylene glycol monomethyl ether, diethylene glycol monoethyl and monomethyl ether, and aromatic alcohols, such as benzyl alcohol and phenoxyethanol, and mixtures thereof.

Further, these compositions may also comprise at least one suitable additive chosen from additives commonly used in compositions for keratinous fibers. Non-limiting examples of the at least one suitable additive include anionic surfactants, cationic surfactants, nonionic surfactants, amphoteric surfactants, fragrances, penetrating agents, antioxidants, sequestering agents, opacifying agents, solubilizing agents, emollients, colorants, screening agents (such as sunscreens and UV filters), preserving agents, conditioning agents, proteins, vitamins, silicones, polymers such as thickening polymers, plant oils, mineral oils, synthetic oils and any other additive conventionally used in compositions for the care and/or treatment of keratinous fibers.

The composition comprising the at least one sugar according to the present invention generally has a pH ranging from 2 to 10. In an embodiment, the pH of the composition ranges from 3 to 8, such as from 5 to 7.

Needless to say, a person skilled in the art will take care to select the at least one suitable additive such that the advantageous properties of the composition in accordance with the invention are not, or are not substantially, adversely affected by the addition(s) envisaged.

The compositions used in the methods and kits of the present invention may also be provided as one-part compositions comprising at least one compound comprising at least one sugar chosen from C₃ to C₅ monosaccharides, and, optionally, at least one additional sugar, or in the form of a multi-component treatment or kit, for example, further comprising at least one relaxing composition. The skilled artisan, based on the stability of the composition and the application envisaged, will be able to determine how the composition and/or multicomponent compositions should be stored and mixed. For example, simple sugars such as C₃ to C₅ monosaccharides are known to be stable at pH levels ranging from 4 to 9. In compositions where the pH range is below or above these levels, e.g., as in a traditional relaxing composition, the sugars would be stored separately and added to the composition only at the time of application.

Thus, the present invention also relates to a kit for relaxing at least one keratinous fiber comprising at least two compartments, wherein a first compartment comprises a first composition comprising at least one sugar chosen from C₃ to C₅ monosaccharides and derivatives thereof; and a second composition comprises a second composition comprising at least one relaxing composition. In one embodiment, at least one composition further comprises at least one additional sugar, different from the at least one sugar chosen from C₃ to C₅ monosaccharides and derivatives thereof.

As previously discussed, the present invention also provides a method for lanthionizing keratinous fibers to achieve relaxation of at least one keratinous fiber comprising (i) applying at least one relaxing composition to at least one keratinous fiber for a sufficient period of time to lanthionize said at least one keratinous fiber; (ii) rinsing the at least one relaxed keratinous fiber; (iii) applying to the at least one relaxed keratinous fiber a composition comprising at least one sugar chosen from C₃ to C₅ monosaccharides and derivatives thereof; and (iv) heating the at least one relaxed keratinous fiber, wherein the at least one sugar is present in an amount effective to prevent reversion of the at least one relaxed keratinous fiber, and further wherein the composition is applied prior to or during heating. In an embodiment, the at least one relaxing composition comprises at least one reducing agent chosen from hydroxide compounds, thiols, sulfites, and derivatives thereof. In an embodiment, the at least one reducing agent is present in a concentration ranging from 4% to 20% by weight of the total weight of said at least one relaxing composition. In an embodiment, the at least one reducing agent is present in a concentration ranging from 7% to 12% by weight of the total weight of said at least one relaxing composition.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. The following examples are intended to illustrate the invention without limiting the scope as a result.

### EXAMPLES

### Example 1

### Comparison of the Effect of 5% D-Xylose Vs. Water on the Percent Reversion of Hair Relaxed with 2.5% Sodium Hydroxide

Virgin African-American (kinky) hair was processed and 2.5% sodium hydroxide for 20 min at room temperature. Next, the sodium hydroxide solution was washed off, the swatch was profusely rinsed with tepid water, and the treatment solution was applied for 5 min at room temperature. The treatment solutions included: water; 1% D-xylose; and 5% D-xylose.

Next, the swatch was briefly rinsed to remove an excess of the treatment solution from the hair surface, blow-dried, and heat was applied in the form of five strokes with a flat iron (130° C.). The heat-treated swatch was rinsed with tepid water, air-dried at 50% RH (25° C.), and maintained for 24 at 90% RH (25° C.). The percent hair straightening (“% straightening”) was determined as follows:

$$\frac{Length of the curly swatch}{Total length of the swatch in the straight configuration} \times 100 ( \% )$$

The percent reversion (“% reversion”)=100%−(% straightening)

TABLE 1

| Hair Type | % Straightening | % Reversion |
|---|---|---|
| Virgin hair | 28.6 | — |
| 2.5% NaOH + water | 85.7 | 14.3 |
| 2.5% NaOH + 1% D-Xylose | 92.8 | 7.2 |
| 2.5% NaOH + 5% D-Xylose | 100 | 0 |

The data of table 1 show that post-treatment with a D-xylose solution prevented curl reversion in the relaxed kinky hair.

### Example 2

### Comparison of the Effect of 5% D-Xylose Vs. Water on the Percent Reversion of Hair Relaxed with Various Concentrations of Sodium Hydroxide

Virgin African-American (kinky) hair was processed with different concentrations of sodium hydroxide for 20 min at room temperature: 1.0%, 1.5%, 2.0%, and 2.5% NaOH, respectively. Next, the sodium hydroxide solution was washed off, the swatch was profusely rinsed with tepid water, and the treatment solution was applied for 5 min at room temperature. The treatment solutions included water and 5% D-xylose.

Next, the swatch was briefly rinsed to remove an excess of the treatment solution from the hair surface, blow-dried, and heat was applied in the form of a flat iron for 1 min (130° C.). The heat-treated swatch was rinsed with tepid water, air-dried at 50% RH (25° C.), and maintained for 24 h at 90% RH (25° C.). The percent hair straightening was determined as in Example 1.

| Hair Type | % Straightening | % Reversion |
|---|---|---|
| Virgin hair | 28.6 | — |
| 1.0% NaOH + water | 42.9 | 57.1 |
| 1.0% NaOH + 5% D-Xylose | 57.1 | 42.9 |
| 1.5% NaOH + water | 71.4 | 28.6 |
| 1.5% NaOH + 5% D-Xylose | 78.6 | 21.4 |
| 2.0% NaOH + water | 80.0 | 20.0 |
| 2.0% NaOH + 5% D-Xylose | 100 | 0 |
| 2.5% NaOH + water | 100 | 0 |
| 2.5% NaOH + 5% D-Xylose | 100 | 0 |

The data show that post-treatment with a D-xylose solution prevented curl reversion in the relaxed kinky hair better than post-treatment with water, even in the case of lower concentrations of sodium hydroxide.

### Example 3

### Effect of pH of D-xylose Solutions

Virgin African-American (kinky) hair was processed with 2.0% sodium hydroxide for 20 min at room. Next, the sodium hydroxide solution was washed off, the swatch was profusely rinsed with tepid water, and the treatment solution was applied for 10 min at 45° C. The treatment solutions included: buffer solution, pH 2.0; buffer solution, pH 6.0; buffer solution, pH 10.0; 5% D-xylose, pH 2.0; 5% D-xylose, pH 6.5; and 5% D-xylose, pH 10. The solutions were buffered using phosphoric acid and sodium hydroxide.

Next, the swatch was briefly rinsed to remove an excess of the treatment solution from the hair surface, blow-dried, and heat was applied in the form of a flat iron for 1 min. (130° C.). The heat-treated swatch was rinsed with tepid water, air-dried at 50% RH (25° C.), and maintained for 24 h at 90% RH (25° C.). The percent hair straightening was determined as in Example 1.

| Hair Type | % Straightening | % Reversion |
|---|---|---|
| Virgin hair | 28.6 | — |
| 2.0% NaOH + water, pH 2.0 | 60.0 | 40.0 |
| 2.0% NaOH + 5% D-Xylose, pH 2.0 | 80.0 | 20.0 |
| 2.0% NaOH + water, pH 6.0 | 70.0 | 30.0 |
| 2.0% NaOH + 5% D-Xylose, pH 6.5 | 90.0 | 10.0 |
| 2.0% NaOH + water, pH 10.0 | 80.0 | 20.0 |
| 2.0% NaOH + 5% D-Xylose, pH 10.0 | 100 | 0.00 |

The data show that post-treatment with a D-Xylose solution prevented reversion of the relaxed hair better than post-treatment with a buffer solution at various pH values.

### Example 4

### Effect of D-xylose Concentration on Hair Relaxed with a Commercial Alkali Relaxer

Virgin African-American (kinky) hair was processed with an alkali relaxer as follows. The commercial cream relaxer was applied to a swatch of hair for 10 min at room temperature. Next, the cream was washed off, the swatch was profusely rinsed with tepid water, and the treatment solution was applied for 5 min. The treatment solutions included water; 1% D-xylose; 3% D-xylose; and 5% D-xylose.

Next, the swatch was briefly rinsed to remove an excess of the treatment solution from the hair surface, blow-dried, and heat was applied in the form of a flat iron for 1 min (175° C.). The heat-treated swatch was rinsed with tepid water, air-dried at 50% RH (25° C.), and maintained for 1 h at 100% RH (25° C.). The percent hair straightening was determined as in Example 1.

| Hair Type | % Straightening | % Reversion |
|---|---|---|
| Virgin hair | 36.4 | — |
| Relaxed hair + water | 46.2 | 53.8 |
| Relaxed hair + 1% D-Xylose | 69.1 | 30.9 |
| Relaxed hair + 3% D-Xylose | 76.0 | 24.0 |
| Relaxed hair + 5% D-Xylose | 78.0 | 22.0 |

The data show that post-treatment with a D-xylose solution prevented reversion of the relaxed hair at various concentrations of D-xylose, as compared to a post-treatment with water.

### Example 5

### Effect of D-xylose Concentration on Hair Relaxed with a Commercial Thioglycolate Relaxer

Virgin African-American (kinky) hair was processed with a commercial thioglycolate cream relaxer as follows. The thioglycolate cream relaxer was applied to a swatch of kinky hair for 15 min at room temperature (reduced hair). Next, the thioglycolate cream was washed off, the swatch was profusely rinsed with tepid water, and a hydrogen peroxide cream was applied for 5 min. The treatment solutions included: water and 5% D-xylose.

The treatment solutions were applied either between the rinsing of the thioglycolate cream relaxer and the application of the hydrogen peroxide cream, or after the application of the hydrogen peroxide cream, respectively. The treatment solutions was applied for 5 min at room temperature. Next, the swatch was briefly rinsed to remove an excess of the treatment solution from the hair surface, blow-dried, and heat was applied in the form of a flat iron for 30 sec (175° C.). The heat-treated swatch was rinsed with tepid water, air-dried at 50% RH (25° C.), and maintained for 1 h at 100% RH (25° C.). The percent hair straightening was determined as in Example 1.

| Hair Type | % Straightening | % Reversion |
|---|---|---|
| Virgin hair | 36.4 | — |
| Reduced hair + Hydrogen Peroxide | 80.0 | 20.0 |
| Reduced hair + Hydrogen | 86.0 | 14.0 |
| Peroxide + 5% D-Xylose | | |
| Reduced hair + 5% D-Xylose + | 100 | 0.00 |
| Hydrogen Peroxide | | |

The data show that post-treatment with a D-xylose solution prevented reversion while using a thioglycolate hair relaxer.

1. A method for preventing reversion of at least one relaxed keratinous fiber comprising:
applying to at least one relaxed keratinous fiber a composition comprising at least one sugar chosen from C₃ to C₅ monosaccharides and derivatives thereof; and
heating said at least one relaxed keratinous fiber,
wherein said at least one sugar is present in an amount effective to prevent reversion of said at least one relaxed keratinous fiber, and
further wherein said composition is applied prior to or during said heating.

2. The method according to claim 1, further comprising wetting said at least one keratinous fiber with water prior to said application.

3. The method according to claim 2, further comprising rinsing said at least one keratinous fiber subsequent to said heating.

4. The method according to claim 1, wherein said at least one relaxed keratinous fiber is heated at 130° C. for 10 seconds.

5. The method according to claim 1, wherein said at least one relaxed keratinous fiber is heated at 180° C. for 3 seconds.

6. The method according to claim 1, wherein said C₃ to C₅ monosacccharides are chosen from pentoses.

7. The method according to claim 1, wherein said pentoses are chosen from aldopentoses and ketopentoses.

8. The method according to claim 7, wherein said aldopentoses are chosen from xylose, arabinose, lyxose, and ribose.

9. The method according to claim 7, wherein said ketopentoses are chosen from ribulose and xylulose.

10. The method according to claim 1, wherein said C₃ to C₅ monosaccharides are chosen from tetroses.

11. The method according to claim 10, wherein said tetroses are chosen from aldotetroses and ketotetroses.

12. The method according to claim 11, wherein said aldotetroses are chosen from erythrose and treose.

13. The method according to claim 11, wherein said aldotetroses are chosen from erythrulose.

14. The method according to claim 1, wherein said C₃ to C₅ monosaccharides are chosen from trioses.

15. The method according to claim 14, wherein said trioses are chosen from aldotrioses and ketotrioses.

16. The method according to claim 15, wherein said trioses are chosen from glyceraldehyde.

17. The method according to claim 15, wherein said trioses are chosen from dihydroxyacetone.

18. The method according to claim 1, wherein said C₃ to C₅ monosaccharides are chosen from furanoses.

19. The method according to claim 1, wherein said at least one sugar is chosen from derivatives of C₃ to C₅ monosaccharides.

20. The method according to claim 19, wherein said derivatives of C₃ to C₅ monosaccharides are chosen from imine derivatives of C₃ to C₅ monosaccharides, hemiacetal derivatives of C₃ to C₅ monosaccharides, hemiketal derivatives of C₃ to C₅ monosaccharides, and oxidized derivatives of C₃ to C₅ monosaccharides.

21. The method according to claim 19, wherein said derivatives of C₃ to C₅ monosaccharides are chosen from oligosaccharides derived from said C₃ to C₅ monosaccharides.

22. The method according to claim 21, wherein said oligosaccharides derived from said C₃ to C₅ monosaccharides are chosen from xylobiose.

23. The method according to claim 19, wherein said derivatives of C₃ to C₅ monosaccharides are substituted.

24. The method according to claim 1, wherein said at least one sugar is xylose.

25. The method according to claim 1, wherein said at least one sugar is present in said composition in an amount ranging from 0.01% to 10% by weight relative to the total weight of the composition.

26. The method according to claim 25, wherein said at least one sugar is present in said composition in an amount ranging from 0.1% to 5% by weight relative to the total weight of the composition.

27. The method according to claim 1, wherein said composition further comprises at least one additional sugar different from said at least one sugar chosen from C₃ to C₅ monosaccharides.

28. The method according to claim 25, wherein said at least one additional sugar is chosen from monosaccharides, oligosaccharides and polysaccharides.

29. The method according to claim 28, wherein said monosaccharides are chosen from hexoses.

30. The method according to claim 29, wherein said hexoses are chosen from allose, altrose, glucose, mannose, gulose, idose, galactose, talose, sorbose, psicose, fructose, and tagatose.

31. The method according to claim 30, wherein said at least one additional sugar is substituted.

32. The method according to claim 27, wherein said at least one additional sugar is present in said composition in an amount ranging from 0.01% to 10% by weight relative to the total weight of the composition.

33. The method according to claim 32, wherein said at least one additional sugar is present in said composition in an amount ranging from 0.1% to 5% by weight relative to the total weight of the composition.

34. The method according to claim 1, wherein said composition is in the form of a liquid, oil, paste, stick, dispersion, emulsion, lotion, gel, or cream.

35. The method according to claim 1, wherein said at least one keratinous fiber is hair.

36. The method according to claim 1, wherein said composition further comprises at least one suitable additive chosen from anionic surfactants, cationic surfactants, nonionic surfactants, amphoteric surfactants, fragrances, penetrating agents, antioxidants, sequestering agents, opacifying agents, solubilizing agents, emollients, colorants, screening agents, preserving agents, conditioning agents, proteins, vitamins, silicones, polymers, plant oils, mineral oils, and synthetic oils.

37. The method according to claim 1, wherein said composition is applied prior to and during said heating.

38. The method according to claim 1, wherein said composition has a pH ranging from 2 to 10.

39. The method according to claim 1, wherein said composition has a pH ranging from 3 to 8.

40. The method according to claim 1, wherein said composition has a pH ranging from 5 to 7.

41. A method for lanthionizing keratinous fibers to achieve relaxation of at least one keratinous fiber comprising:
applying at least one relaxing composition to at least one keratinous fiber for a sufficient period of time to lanthionize said at least one keratinous fiber;
rinsing said at least one relaxed keratinous fiber;
applying to at least one relaxed keratinous fiber a composition comprising at least one sugar chosen from C₃ to C₅ monosaccharides and derivatives thereof; and
heating said at least one relaxed keratinous fiber,
wherein said at least one sugar is present in an amount effective to prevent reversion of said at least one relaxed keratinous fiber, and
further wherein said composition is applied prior to or during said heating.

42. A method according to claim 41, wherein said at least one relaxing composition comprises at least one reducing agent chosen from hydroxide compounds, thiols, sulfites, and derivatives thereof.

43. A method according to claim 42, wherein said at least one reducing agent is present in a concentration ranging from 4% to 20% by weight of the total weight of said at least one relaxing composition.

44. A method according to claim 43, wherein said at least one reducing agent is present in a concentration ranging from 7% to 12% by weight of the total weight of said at least one relaxing composition.

45. A kit for relaxing at least one keratinous fiber comprising at least two compartments,
wherein a first compartment comprises at least one sugar chosen from C₃ to C₅ monosaccharides and derivatives thereof; and
wherein a second compartment comprises at least one relaxing composition.

46. A kit according to claim 45, wherein at least one of said at least two compartments further comprises at least one additional sugar, different from said at least one compound.
